(19) United States
(12) Patent Application Publication
(10) Pub. No.: US 2022/0362047 A1
(43) Pub. Date: Nov. 17, 2022

(54) FOOT BRACE

(71) Applicant: Sams Sebastian Nataresa, Palm Desert, CA (US)

(72) Inventor: Sams Sebastian Nataresa, Palm Desert, CA (US)

(21) Appl. No.: 17/487,963

(22) Filed: Sep. 28, 2021

Related U.S. Application Data
(60) Provisional application No. 63/258,553, filed on May 13, 2021.

(51) Int. Cl. A61F 5/01

(52) U.S. Cl. CPC A61F 5/0127; A61F 2005/0172

(57) ABSTRACT

A foot brace to manage foot supination includes a planar member, the planar member has a proximal end and a distal end, an upper portion, a middle portion, and a lower portion. The middle portion can include an opening for the heel to protrude. A leg fastener at the proximal end of the planar member allows securing the foot brace to the leg. A foot strap at the distal end allows securing the foot brace to the midfoot or the forefoot. On an inner surface of the planar member adjacent to the distal end can be provided multi-tier cushions pads, wherein the multi-tier cushion pads can put an upward force on the arch of the foot when the foot with the foot brace is placed on the floor. The number of cushion pads can be changed for optimum stabilization effect in foot supination.

### CROSS-REFERENCE TO RELATED APPLICATIONS

This application claims priority from the U.S. provisional patent application Ser. No. 63/258,553, filed on May 13, 2021, which is incorporated herein by reference in its entirety.

### FIELD OF INVENTION

The present invention relates to a foot brace, and more particularly, the present invention relates to a foot brace to manage foot supination.

### BACKGROUND

A number of physical ailments can be due to muscle imbalances, upper body and lower body crossed syndrome, and includes foot supination. Supination and pronation are parts of a stride. Excessive supination, referred to herein as foot supination, happens when a person doesn't use the proper muscles to walk correctly. This may be due to several reasons, such as highly arch feet. Foot supination can cause several ailments, such as pain and discomfort during a walk and even at rest.

A need is appreciated for a device that can manage foot supination. A need is appreciated from a device that can help people with high arch to walk painlessly.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

It is therefore a principal object of the present invention directed to a foot brace that can help manage foot supination.

It is another object of the present invention that the foot brace allows a person with supinated foot to walk painlessly.

It is still another object of the present invention that the foot brace can easily adjust to variable heights of arches in different people.

It is a further object of the present invention that the foot brace can easily adjust to different sizes of feet.

It is still a further object of the present invention that the foot brace is economical to manufacture.

In one aspect, disclosed is a foot brace to manage foot supination. The foot brace includes a planar member, the planar member has a proximal end and a distal end. The planar member can include an upper portion, a middle portion, and a lower portion. The upper portion of the planar member can engage against a rear portion of a leg adjacent to an ankle, the middle portion can include an opening for the heel to protrude, and the lower portion can engage against the midfoot and optionally to the forefoot. A leg fastener at the proximal end of the planar member allows securing the foot brace to the leg. A foot strap at the distal end allows securing the foot brace to the midfoot or the forefoot. The opening in the middle portion may allow the heel to protrude through the opening for stabilizing the ankles. The planar member can have an inner surface that comes in contact with the skin and an opposite outer surface that is exposed to environment. Over the inner surface adjacent to the distal end can be provided multi-tier cushions pads, wherein a top cushion pad of the multi-tier cushions pads can come in contact with the foot and may be made from a skin-friendly material. The top cushion pad of the multi-tier can remain at the top while the lower cushion pads can be interchanged. For example, each cushion pad can be fastened one above another on the inner surface of the planar member and the top cushion pad can remain same. The multi-tier cushion pads can put an upward force on the arch when the foot wearing the foot brace is placed on the floor. The number of cushion pads can be changed for optimum stabilization effect in foot supination.

In one case, the cushion pads can be fastened to each other through a hook and loop fastener, The hook and loop fastener has a male mating member and a female mating member, wherein one side of the cushion pad can be provided with the male mating member and the other side can be provided with the female mating member. The top cushion pad can have the hook and loop fastener only on its bottom side.

In one case, a base cushion pad can be bonded to the inner surface of the planar member and the other cushion pads can be fastened one above another over the base cushion pad.

In one aspect, the inner surface of the planar member can have a patch of the hook and loop fastener to which the cushion pads can be fastened one above another to form the multi-tier cushion pads.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a perspective view of the foot brace, according to an exemplary embodiment of the present invention.

FIG. 2 shows the foot brace folded, according to an exemplary embodiment of the present invention.

FIG. 3 shows the foot brace worn in the foot according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an”, and “the” are intended to include the plural forms as well, unless the context clearly indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

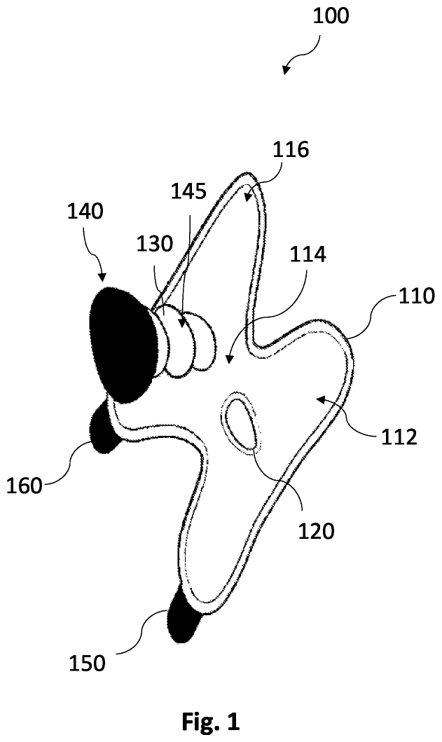

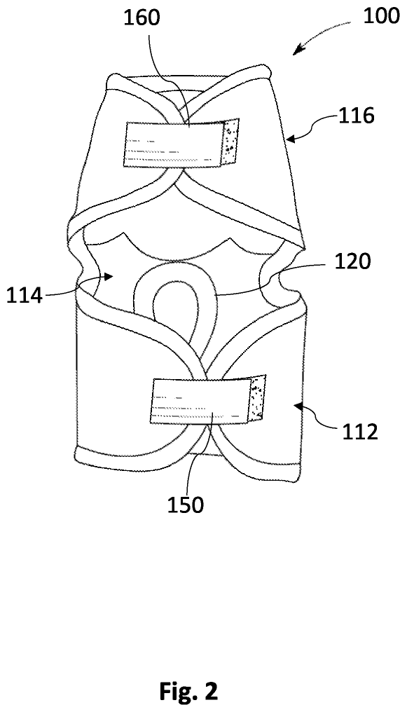

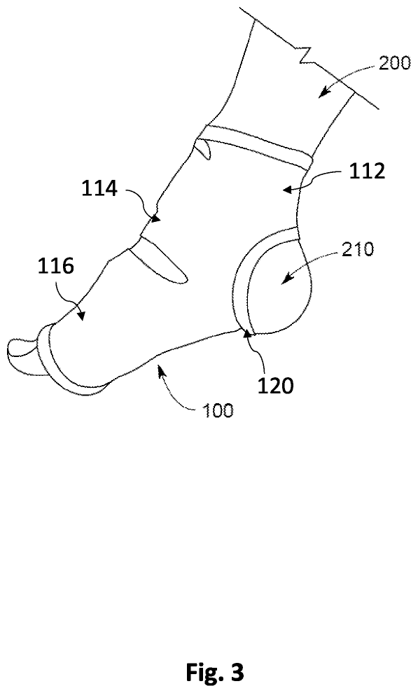

Disclosed is a foot brace that can be applied to a foot for managing foot supination. The disclosed foot brace can apply an upward force to the arch when the foot wearing the foot brace is placed on the floor. A person suffering from foot supination can wear the disclosed foot brace to walk painlessly. The disclosed foot brace by applying force against the arch tends to balance the muscles while supination resulting in a balanced stride. Referring to FIG. 1 which shows an exemplary embodiment of the foot brace 100. FIG. 2 shows the foot brace 100 folded with the leg fastener and the foot fastener coupled. FIG. 3 is an environmental diagram showing the foot brace 100 worn in a foot 200. The foot brace 100 can include a planar member 110 that can have a top surface and a bottom surface. The top surface comes in contact with the skin and can be made from skin-friendly material. The bottom surface can be made from flexible but durable material. In one case, the bottom surface can have a layer of water-resistant material. The planar member 110 can have a proximal end and a distal end. The planar member 110 can have an upper portion 112, a middle portion 114, and a lower portion 116. The upper portion 112 can cover a rear portion of the leg nearby the ankle. The middle portion 114 can have an opening for the heel to protrude. FIG. 3 shown the foot brace 100 applied on a foot 200. Heel 210 can be seen protruding from the opening 120 in the middle portion 114 of the planar member 110. The opening 120 by allowing the heel to produce can stabilize the ankle while walking. The lower portion 116 can extend up to the forefoot.

To secure the foot brace 100 on the foot, a leg strap 150 can be provided which can wrap around the leg adjacent to the ankle. The leg strap can include a hook and loop fastener that includes a male mating member on one end and a female mating member on the opposite end, wherein the male mating member can couple to the female mating member for fastening the leg strap. The hook and loop fastener may allow adapting the leg strap to legs of different sizes. It is to be understood that hook and loop fasteners may be a preferred fastener, however, any other fastener known to a skilled person for fastening two ends of strap(s) is within the scope of the present invention. Similarly, a foot strap 160 can also be provided at the distal end to secure the foot brace 100. The foot strap 160 can wrap around the midfoot or the forefoot. FIG. 2 shows the foot brace 100 folded wherein two ends of the leg strap 150 and two ends of the foot strap 160 are shown coupled.

On an area on the inner surface of the planar member and adjacent to the distal end can be seen multi-tier cushion pads 130. The multi-tier cushion pads can include a top cushion pad 140 and lower cushion pads 145. The cushion pads including the top cushion pad and the lower cushion pads can be fastened one above another through fasteners, such as hook and loop fasteners, to form the multi-tier cushion pads. The fastener can have a male mating member and a female mating member. Each lower cushion pads can be provided with a male mating member on one side and a female mating member on the opposite side. The planar member can be provided with a base cushion pad bonded to an area on the inner surface of the planar member, wherein the exposed surface of the base cushion pad can have the mating member of the fastener. Alternatively, a patch of the mating member can be bonded to the area and the cushion pads can be fastened one above another over the base pad or the patch. The fastener may allow the lower cushion pads to be added one above another, wherein the number of cushion pads can be increased or decreased. The top cushion pad can have the mating member of the fastener only at the bottom side wherein the top surface of the top cushion pad can have a layer of skin-friendly material.

The multitier cushion pads can put an upward force on the arch when the foot with the foot brace is placed on a floor, such as upon standing or walking. By putting pressure against the arch, the multitier cushion pads can support the arch balancing the muscles and thus reducing the foot supination. Both the stabilization of the ankle by the protruded heel and an upward force on the arch tends to balance the stride by managing the foot supination. The height of the multitier support can be adjusted by increasing or decreasing the number of the lower cushion pads. For example, a user can wear the foot brace and try walking and can check the comfort. The user can add more cushion pads or remove an existing cushion pad for the desired comfort.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

1. A foot brace configured to be worn over a foot for managing foot supination, the foot brace comprising:
a planar member, the planar member has a proximal end and a distal end, the planar member has an upper portion, a middle portion that has an opening for a heel of the foot to protrude through the opening, and a lower portion configured to engage to a midfoot and optionally to a forefoot;
a leg strap at the proximal end of the planar member configured to wrap around a leg adjacent to an ankle;
a foot strap at the distal end of the planar member and configured to wrap around a midfoot or a forefoot; and
multi-tier cushion pads extending upwards from an area of an inner surface of the planar member adjacent to the distal end, wherein the multi-tier cushion pads configured to put an upward force on an arch of the foot when the foot wearing the foot brace is placed on ground;
wherein the multi-tier cushion pads comprise a top cushion pad and one or more lower cushion pads,
wherein the top cushion pad and the one or more lower cushion pads are fastened one above another through a fastener, wherein a number of the one or more lower cushion pads can be varied,
wherein the fastener has a male mating member and a female mating member, the male mating member configured to removably couple with the female mating member, wherein each cushion pad of the one or more lower cushion pads has the male mating member on one side and the female mating member on opposite side.

2. (canceled)

3. (canceled)

4. The foot brace according to claim 1, wherein the leg strap has a hook and loop fastener.

5. The foot brace according to claim 4, wherein the foot strap has a hook and loop fastener.

6. (canceled)

7. (canceled)

8. The foot brace according to claim 1, wherein the top cushion pad has a skin friendly layer on a top surface and a mating member of the fastener on a bottom surface.

9. The foot brace according to claim 8, wherein the foot brace further comprises a base cushion pad bonded to the area of the planar member, a mating member of the fastener provided on a top surface of the base cushion pad.

10. The foot brace according to claim 8, wherein the foot brace further comprises a patch bonded to the area of the planar member, a mating member of the fastener provided on a top surface of the patch, wherein the patch and the one or more lower cushion pads are different.

11. A method for managing foot supination, the method comprising the steps of:
providing a foot brace comprising:
a planar member, the planar member has a proximal end and a distal end, the planar member has an upper portion, a middle portion that has an opening for a heel of a foot to protrude through the opening, and a lower portion configured to engage to a midfoot and optionally to a forefoot;
a leg strap at the proximal end of the planar member configured to wrap around a leg adjacent to an ankle;
a foot strap at the distal end of the planar member and configured to wrap around a midfoot or a forefoot; and
multi-tier cushion pads extending upwards from an area of an inner surface of the upper portion, wherein the multi-tier cushion pads configured to put an upward force on an arch of the foot when the foot wearing the foot brace is placed on ground,
wherein the multi-tier cushion pads comprise a top cushion pad and a plurality of lower cushion pads,
wherein the top cushion pad and the one or more lower cushion pads are fastened one above another through a fastener, wherein a number of the plurality of lower cushion pads can be varied,
wherein each cushion pad of the plurality of lower cushion pads has mating members of the fastener on opposite sides, and
wearing the foot brace over the foot, such that the heel of the foot protrudes from the opening in the middle portion and the multi-tier cushion pads push against the arch of the foot;
removing a cushion pad from the plurality of lower cushion pads; and
upon removing, wearing the foot brace over the foot.

12. (canceled)

13. (canceled)

14. The method according to claim 11, wherein the leg strap has a hook and loop fastener.

15. The method according to claim 14, wherein the foot strap has a hook and loop fastener.

16. (canceled)

17. (canceled)

18. The method according to claim 11, wherein the top cushion pad has a skin friendly layer on a top surface and a mating member of the fastener on a bottom surface.

19. The method according to claim 18, wherein the foot brace further comprises a base cushion pad bonded to the area of the planar member, a mating member of the fastener provided on a top surface of the base cushion pad.

20. The method according to claim 18, wherein the foot brace further comprises a patch bonded to the area of the planar member, a mating member of the fastener provided on a top surface of the patch, wherein the patch and the one or more lower cushion pads are different.

21. The foot brace according to claim 1, wherein the planar member has a left long side and a right long side, each of the left long side and the right long side is arched inwards to form an upper broad portion, a lower broad portion, and a middle narrow portion of the planar member.

22. The method according to claim 11, wherein the planar member has a left long side and a right long side, each of the left long side and the right long side is arched inwards to form an upper broad portion, a lower broad portion, and a middle narrow portion of the planar member.
